(19) United States
(12) Patent Application Publication
(10) Pub. No.: US 2003/0032066 A1
(43) Pub. Date: Feb. 13, 2003

(54) Protein-protein interaction map inference using interacting domain profile pairs

(76) Inventors: Pierre Legrain, Paris (FR); Jerome Wojcik, Paris (FR); Vincent Schachter, Paris (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/100,841

(22) Filed: Mar. 19, 2002

Related U.S. Application Data
(60) Provisional application No. 60/277,021, filed on Mar. 19, 2001.

(51) Int. Cl. G01N 33/53; G06G 7/48; G06G 7/58

(52) U.S. Cl. 435/7.1; 701/19; 703/11

(57) ABSTRACT

Disclosed is a technique to predict protein-protein interaction maps across organisms called an “interacting-domain profile pair” method. The method uses a high-quality protein interaction map with interaction domain information as input to predict an interaction map in another organism. It combines sequence similarity searches with clustering based on interaction patterns and interaction domain information. Results can be compared with predictions from a naive inference method based only on full-length protein sequence similarity. This domain-based method can eliminate a significant amount of false-positives compared to methods that are the consequences of multi-domain proteins; as well as increase the sensitivity by identifying new potential interactions.

The present invention claims priority from U.S. provisional application serial No. 60/277,021, the contents of which are incorporated herein by reference.

### BACKGROUND

With the completion of the full genome sequence for several model organisms, new approaches are emerging to comprehensively characterize the function of gene products. In these so-called “functional proteomics” approaches, large-scale assays on the complete set of proteins of a given organism (the proteome) enable the study of the function of proteins in their context, rather than individually.

The recent emergence of high-throughput techniques to systematically identify physical interactions between proteins has opened new prospects. Not only can an important part of what is now referred to as the “function” of a protein be characterized more precisely through its interactions, but networks of interacting proteins also extend this purely local view of function by providing a first level of understanding of cellular mechanisms. In short, protein interaction maps can provide detailed functional insights on characterized as well as yet uncharacterized proteins, along with an information base for the identification of biological complexes and metabolic or signal transduction pathways (for review, see Walhout and Vidal 2001).

On the experimental front, high-throughput techniques derived from the yeast two-hybrid system have been used to build protein interaction maps for several organisms, including Saccharomyces cerevisiae (Fromont-Racine, Rain et al. 1997; Fromont-Racine, Mayes et al. 2000; Ito, Tashiro et al. 2000; Uetz, Giot et al. 2000), Caenorhabditis elegans (Walhout, Sordella et al. 2000), the HCV (Flajolet, Rotondo et al. 2000) and vaccinia (McCraith, Holtzman et al. 2000) viruses, and recently the Helicobacter pylori bacteria (Rain, Selig et al. 2001).

As initial in silico exploitations of these experimentally derived interaction maps, algorithms aimed at assigning function to uncharacterized gene products have been proposed. Their underlying principle is “guilt by association”; i.e., the function is assigned to a protein by transposing existing annotations from its interacting partners. This approach relies heavily on the completeness of the interaction map and on the quality of functional annotations. First attempts of this sort were performed recently on the S. cerevisiae protein interaction map (Fellenberg, Albermann et al. 2000; Schwikowski, Uetz et al. 2000).

On the computational front, protein linkage maps have also been predicted ab initio using algorithms based on sequence data from completely sequenced genomes, such as the “Rosetta Stone”/“gene fusion” method (Enright, lliopoulos et al. 1999; Marcotte, Pellegrini et al. 1999), the “phylogenetic profiles” method (Pellegrini, Marcotte et al. 1999), the “gene neighbor” method (Dandekar, Snel et al. 1998; Overbeek, Fonstein et al. 1999), or the mRNA expression level correlation method (Eisen, Spellman et al. 1998). Links predicted by these in silico approaches hint at correlated function, with a part corresponding to actual physical interactions. Each approach shows an a priori bias corresponding to the biological hypothesis underlying the prediction algorithm; e.g., two proteins interact if their genes were fused in an ancestor genome. Comparison with experimental data confirms this bias, and also shows an increase in predictive power when several independent sources of data and different algorithms are combined (Marcotte, Pellegrini et al. 1999) (See, Eisenberg, Marcotte et al. 2000, for review).

Classical attempts to predict functional properties of proteins across organisms typically involve two major conceptual steps. The first conceptual step involves the establishment of a correspondence between proteomes, i.e., a function that associates to each protein of the source organism a set of proteins in the target organism. The second conceptual step involves the transport of the property of interest along that correspondence.

In comparative genomics approaches, the focus is on transporting functional annotations of individual proteins to putative orthologs (i.e., exact functional counterparts) in the other organism; it is thus important to distinguish orthologs from paralogs (i.e., homologous genes that have arisen through a gene duplication and have evolved in parallel in the same organism). The correspondence is thus by construction one-to-one, in agreement with this “atomic” notion of function.

The problems associated with the known classical methods cited in the art to predict functional properties of proteins across organisms is that these methods rely on the full-length protein sequence in the comparison. Thus, the full-length protein sequence of the source organism is compared to the full-length protein sequence in the target organism. One method deducible from the prior art is the “naive method”. From a theoretical point of view, the naive method has two major a priori weaknesses, namely:

(1) it does not take into account the fact that interactions occur between protein domains rather than full proteins, nor does it exploit the domain information in the source map; and

(2) it does not fully exploit the network structure of the source map: indeed, this method treats in the same way a list of unconnected interactions and a densely connected interaction map. It is clear, however, that knowing that several different proteins interact with x through homologous domains is a better support for prediction than just knowing of one such interaction. Similarly, this method does not exploit the fact that the property to be inferred is a property of pairs of proteins rather than of individual proteins. Consequently, there are many false positives in the naive method. Hence, the protein interaction maps based on the use of the naive method may be quite inaccurate.

Another expectation of proteomics is the identification and the annotation of new profiles, the present invention allows the definition of new annotated profiles and the annotation of already existing profiles such as those listed in the Interpro data bases (Apmeiler et al 2000).

### SUMMARY OF THE PRESENT INVENTION

Thus, the present invention relates to overcoming the absence in the prior art of techniques of predicting a protein-protein interaction map.

One aspect of the present invention relates to a method for obtaining a predicted protein-protein interaction map across organisms, comprising:

(1) creating an intermediary domain cluster interaction map e.g., from at least one of a connectivity link (I-link) and a sequence similarity link (S-link) taken from at least one of source map, a protein expression profile and an art annotation;

(2) optionally building a profile for each interacting domain cluster from said intermediary domain cluster interaction map;

(3) searching for similarities between said profile for each interacting domain cluster and in a target organism;

(4) creating a correspondence between the intermediary domain cluster interaction map and the target organism from said similarities; and

(5) predicting a target protein interaction map along the correspondence.

Another aspect of the present invention is directed to a method of predicting a target organism protein interaction map from a source organism protein interaction map, comprising:

(1) comparing each target organism protein sequence with each source organism protein; and

(2) transporting an interacting property of two source organism proteins along two target organism proteins showing significant similarities with the two interacting source organism proteins.

Yet another aspect of the present invention provides a method for predicting an organism protein interaction map from a source organism protein interaction map by comparing each target organism protein sequence with each interacting domain of a source organism protein specifically involved in an interaction.

A further aspect of the present invention provides a protein-protein interaction map (PIM®) obtained by the above processes, as well as a record or report of the protein-protein interaction map e.g., in electronic, paper or digital form. A Predicted Biological Score (hereinafter referred to as PBS®) can be provided at the time of generating the Protein Interaction Map or PIM®. This treatment permits the knowledge of the probability that the deduced interaction exists effectively in nature.

In yet a further aspect, the present invention provides a method wherein the combination of sequence and interaction information allows the identification of interacting domain profiles, “flexible patterns” of sequence correlated to physically interacting structures that enhance the prediction sensitivity. As an additional feature, these profiles that are a flexible sequence pattern also represent new potential binding motifs.

Several advantages are associated with the present invention. For example, it utilizes interacting domains in order to reduce the false-positive rate induced when predicting an interaction from multi-domain proteins. In addition, it exploits the network structure of the reference interaction map with sequence data from the interaction domains to increase prediction sensitivity and specificity.

### BRIEF DESCRIPTION OF THE DRAWINGS

FIG. 1 is a flow chart representation of the Interacting Domain Protein Profile (IDPP) method of the present invention in comparison with the naive method.

FIG. 2 is a schematic representation of the clustering of interacting domains (ID) into n-Sic. The interacting domains that interact with a given protein A in the interaction map (a) are connected in terms of I-links (b) and S-links (c). An I-link between Bᵢ and Bⱼ means that Bᵢ and Bⱼ interact with the same region of A. A S-link means Bᵢ and Bⱼ share the same sequence similarity. The Interacting Domains are then clustered into n-Sic by determining cliques (sub-graphs where each vertex is connected to all others) both in terms of I-links and S-links (d).

FIG. 3 is the definition of the Interacting Domain Profile Pairs. A pair of n-SIC (X-Y) defines an ID profile pair (IDPP) if the proportion of interactions between IDs of X and IDs of Y, compared to the total number of possible interactions (xy), is greater than a given threshold T. This Figure illustrates a x:y IDPP.

FIG. 4 is a schematic representation of the prediction of gyrA homodimerization in E. coli.

FIG. 5 is a schematic diagram illustrating that the IDPP algorithm takes into account both the similarities of sequence and connectivity within the interaction network in order to build interacting domain profiles; i.e., a consensus sequence for interacting domains which need to be conserved in the target organism in order to transfer and predict a given interaction.

FIG. 6 is a schematic diagram of a partial protein-protein interaction map of Campylobacter jejuni and Escherichia coli. Also, the source interaction network from H. pylori that was used to infer these maps is illustrated.

### DETAILED DESCRIPTION OF THE PREFERRED EMBODIMENTS

As used herein, the term “homologs” means structurally similar genes within a given species, while “orthologs” are functionally equivalent genes from a given species or strain, as determined, for example, in a standard complementation assay. Thus, a polypeptide of interest can be used not only as a model for identifying similar genes in given strains, but also to identify homologs and orthologs of the polypeptide of interest in other species, The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein an “ID” means an Interacting Domain (ID) and is a polypeptidic fragment of a protein, such fragment being involved in the interaction between the protein and another protein. An ID may be identified, for example, with the yeast two-hybrid system as described in WO 00/66722; an example of an ID may be a bait or a SID®.

As used herein the “Interacting Domain Profile Pair (IDPP) method” is closely related and has been adjusted to the data generated with the two-hybrid system, but may be applied to any method leading to the identification of protein-protein interactions and to the determination of specific domains involved in these interactions such as 2-dimensional gel results, protein chips, BRET technology, mass spectroscopy and the like.

As used herein the “sticky” preys or “sticky domains” is a SID® that is found in an unexpectedly high number of screens and corresponds to a strongly connected prey vertex in the PIM®.

As used herein the term “PIM®” means a protein-protein interaction map. This map is obtained from data acquired from a number of separate screens using different bait polypeptides and is designed to map out all of the interactions between polypeptides.

As used herein, “PBS®” means a Predicted Biological Score and is a reliability score for protein-protein interactions derived from yeast two-hybrid screenings described in WO 99/42612, which is incorporated herein by reference. The aim of the PBS® computation is to make the generated PIM®s more sensitive by filtering out false positives and rescuing false negatives.

The PBS® is computed as a combination of one or more “component scores” which are the internal PBS and zero and one or several external PBS's. The internal PBS is computed using results obtained from the yeast two-hybrid screenings. The computation features two steps.

The first step is the local internal PBS, derived from each individual screen and is a reliability score for bait-to prey oriented interactions. It is based on a statistical model of the experimental process, modified by some biological expertise post-processing. For each screen, positively selected fragments are clustered in order to define Selected Interacting Domains (SID®s). The SID®s define patterns for potentially matching fragments a posteriori. Thus, the probability of randomly selecting the fragments that define an interacting SID® can be computed from the fragment distribution in the initial prey library. The local internal PBS is computed as the noise/signal ratio of the observed results to this background probability, expressed as an E-value (expectation value) probability ranging from 1 to 0. An E-value close to 1 (100%) means that the interaction is very probably an artifact; whereas an E-value close to 0 means that it is probably biologically relevant. The biological expertise modifies this initial score by applying strategies to deal with specific cases such as obtaining antisense, intergene or out-of frame fragments.

The global internal PBS takes into account the whole PIM® and gathers oriented interactions yielding local internal PBS to filter out the “sticky” preys and to score non-oriented protein-protein interactions. First, bait and SID®(prey) fragments representing the same region are clustered together. Second, connectivity patterns are examined to detect abnormally connected regions. If sticky domains are detected, they are discarded. Unsuccessful screens/baits, leading to oriented interactions with local PBSs close to 1 (minimum) are discarded as well.

The external PBS are interaction scores derived from external information such as a SID® sequence analysis, bibliographical data, in vivo expression assays, additional biological validations or 2-hybrid data from external sources. External data are automatically obtained from “mining” public databases.

The final PBS is thus presented as an unique score resulting from the combination of the internal PBS and each of the external PBS available for a given protein-protein interaction. However, the trace of each intermediary PBS is kept to help interpretation. Moreover, in order to facilitate the understanding and the usability as selection criteria in the PIM Rider®, the PBS's are regrouped into five (5) categories from A (high significance) to E (low significance).

As used herein, the term “PIM Rider®” is a database and computer system that provides a direct access to specific host data repository that stores the various PIM®s. This repository is generated from a computerized production environment that supports and automates all the activities of the host production facilities. These activities include, but are not limited to, management and follow up of the production, initiation of biotechnological programs, access to all useful information about proteomes under study, definition of processes and biotech/bioinformatics operations required by the technologies, enforcement of protocols, data acquisitions and organized storage, automate interface, plate and biological material physical storage information, quality control, routine analysis of results, visualization of results, computation of PBS®s, storage of SID®s and fragments, reanalysis of results when new external information is available, data mining, delivery of analysis results for the system and the like.

The system software follows a multi-layered web architecture, wherein each layer is able to be physically distributed on separate hardware and scaled independently, an (object-relational) data base management system, a data base object and structure, an object-oriented language (for example, Java) to implement the business-object layer, the SQL language to access the data bases, a middleware layer (currently implemented with Java Server Page (JSP)) to process a user's request and to generate on the fly the HTML pages of the user interface, a set of applications to perform specific tasks on Host servers, a set of applications and applets to perform specific tasks on the client's machine and a set of visualization and display screens accessible through a WWW browser.

As used herein “cliques” refers to sub-graphs where each vertex is connected to all others.

As used herein, the terminology “Interacting Domain Profile” means a characterization of a number, for example, between 2 and 10, 2 and 7 and on the average between 2 and 3, of ID sequences for several organisms and these sequences are not linked to one organism.

By “clustering of domains” is meant the grouping of different protein domains. For example, criteria for clustering domains may be the property (i) to interact with the same region of the same partner, (ii) to show sequence similarity or a combination of (i) and (ii).

To determine the score of sequence similarity of two amino acids sequences or two nucleic acid sequences, the sequences are aligned for optimal comparison with alignment methods (see above). For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

For example, one score may be the percent identity between the two sequences is a function of the number of identical positions shared by the sequences. Hence % identity=number of identical positions/total number of overlapping positions×100.

In this comparison the sequences can be the same length or may be different in length. Optimal alignment of sequences for determining a comparison window may be conducted by the local homology algorithm of Smith and Waterman (J. Theor. Biol., 91 (2) pgs. 370-380 (1981), the homology alignment algorithm of Needleman and Wunsch, J. Miol. Biol., 48(3) pgs. 443-453 (1972), the search for similarity via the method of Pearson and Lipman, PNAS, USA, 85(5) pgs. 2444-2448 (1988), computerized implementations of these algorithms (GAP, BESTFIT, FASTA and TFASTA in the Wisconsin Genetics Software Package Release 7.0, Genetic Computer Group, 575, Science Drive, Madison, Wis.) by inspection, or by BLAST (Atschul at al 1990). Each method provides a comparison score that characterizes the sequence similarity.

The best alignment (i.e., resulting in the highest percentage of identity over the comparison window) generated by the various methods is selected.

The term “sequence identity” means that two polynucleotide or polypeptide sequences are identical (i.e., on a nucleotide by nucleotide or an amino acid by amino acid basis, respectively) over the window of comparison. The protein-protein interaction map of the “source organism” or “reference organism” is known and gives the starting set of data to perform the IDPP method according to the present invention.

On the contrary, the protein-protein interaction map of the “target organism” is yet not known and its identification represents the goal to achieve by using the IDPP method.

By “flexible sequence pattern” it is meant a polypeptide sequence for which each position is represented by a frequency law of the different amino acids that could occur in this position, rather than a given amino acid.

A “significant sequence similarity” means, for example, a SW (Smith Waterman) score>50, or a E value<10⁻⁴. Significant sequence similarity and homolgous sequences are used interchangeably. The present invention relates to a computational approach that predicts the protein interaction map of a target organism from a large-scale “reference” interaction map (of a source organism) that includes selected interaction domain information. The selected interaction domain information was obtained using a yeast two-hybrid system as described in WO 99/42612 or WO 00/66277. WO99/42612 describes a method that permits the screening of prey polynucleotides with a given bait polynucleotide in a single step due to the cell to cell mating strategy between haploid yeast cells. The SID® polynucleotides are then identified by comparing and selecting the intersection of every isolated fragment that are included in the same polypeptide as described in, for example Szabo et al Curr Opin Struct Biol 5 pgs. 699-705 (1995).

The present method referred to as the “Interacting Domain Profile Pairs” (IDPP) approach is based on a combination of interaction data and sequence data, and uses a combination of homology searches and clustering.

The method of the present invention can be used to deduce or infer a protein interaction map from a variety of organisms, such as bacteria, yeasts, fungi, insects, nematodes, mammalians, plants and the like. In the present invention an Escherichia coli protein-protein interaction map is inferred from a Helicobacter pylori reference interaction map. H. pylori was chosen as the source organism to exemplify the present invention because its published protein interaction map contains the largest set of reliable experimental interactions, and includes information on interaction domains (Rain, Selig et al. 2001). However, the present invention is not limited to these two organisms.

The Interacting Domain Profile Pairs (IDPP) prediction results are compared to results obtained using a naive interaction map prediction method based only on full-length protein sequence similarities, similar to techniques used for functional inference between putative orthologs in a number of comparative genomics studies (Bansal 1999) (for review see (Bork, Dandekar et al. 1998)) and to an earlier attempt at inferring pathways across organisms (Karp, Ouzounis et al. 1996).The Interacting Domain Profile Pairs (IDPP) method is shown to both eliminate a significant number of false positives of the naive method by addressing the issue of multi-domain proteins, and to exhibit increased sensitivity by predicting additional domain-based interactions.

The present invention thus relates to a method for obtaining a predicted protein-protein interaction map across organisms the method comprising:

(1) creating an intermediary domain cluster interaction map for a source organism from a connectivity link (I-link) and/or other proteomic data such a sequence similarity link (S-link) from a source map or a protein expression profile, or an annotation from the art;

(2) optionally building a profile for each selected interacting domain cluster from said intermediary domain cluster interaction map;

(3) searching for similarities between said cluster for each interacting domain cluster and in a target organism;

(4) creating a correspondence between the intermediary domain cluster interaction map and the target organism from said similarities; and

(5) predicting a target protein interaction map along the correspondence.

Prior to giving greater detail concerning the present invention and in order to facilitate the description and motivation of the two methods that were used to predict a protein interaction map, the following notations are used.

(1) a proteome P is represented as a set of proteins {p₁, . . . , pₘ}.

(2) a set of domains of P is a set Dₚ={d₁, . . . , dₙ} such that each dᵢ belongs to at least one protein of P. Interacting domains (IDs) are particular instances of domains.

(3) an interactome I is represented as a set of interactions {i₁, . . . , iₙ}, each interaction i connecting a pair (pᵢ, pⱼ). In addition, an interaction can be regarded as a link between SIDs (dᵢ, dⱼ), with domains dᵢ and dⱼ belonging to pᵢ and pⱼ, respectively.

(4) a protein interaction map M=(P, I) is represented as a graph were the edges are the interactions of I that connect the vertex proteins of P.

For convenience, it should be noted that M_{S}=(P_{S}, I_{S}) the Source, and M_{T}=(P_{T}, I_{T}) the Target protein interaction map.

To obtain the predicted interaction map of the present invention the Interacting Domain Profile-Pair (IDPP) method is used. The IDPP method first creates an intermediary “Domain cluster interaction map” (MD_{S}). MD_{S} vertices are obtained by clustering Interacting Domains according to two criteria of which are connectivity (I-links) and sequence similarity (S-links) that are derived from the source interaction map. An MDs interaction between two Interacting Domain clusters is created when enough interactions exist between members of the cluster pair. Generally between about 50% and 100% of interactions is when enough interactions exist between members of the cluster pair. A profile is then built for each Interacting Domain cluster and used to screen; (i.e., to compare the profile with the entire protein sequences of) P_{T} and create a MD_{S} and M_{T} correspondence or MD_{S} and P_{T} correspondence. The target protein interaction map is then predicted along this latter correspondence.

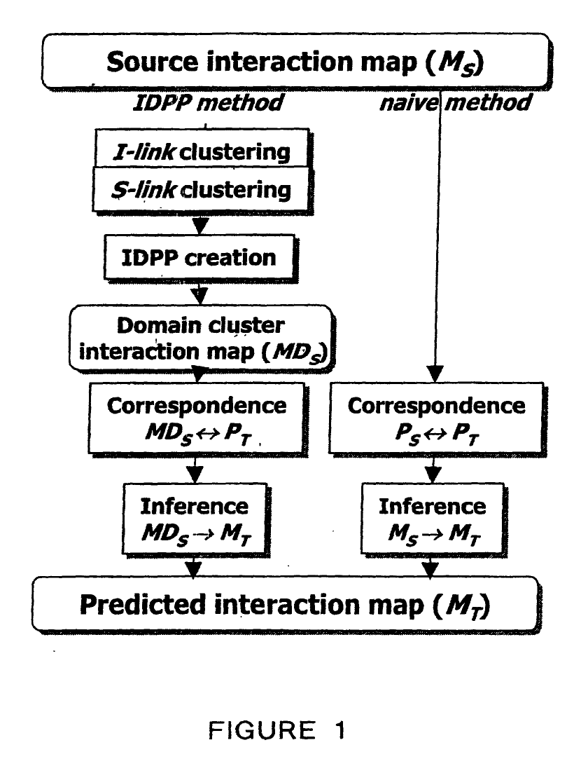

The IDPP algorithm predicts a protein interaction map on a target proteome P_{T} from a source map M_{S}. In contrast to the naive method, however, it fully exploits the properties of the available instance of M_{S}, namely the availability of domain information for each interaction and the fact that for a given ID domain d of a protein x, the protein interaction map will typically provide several instances of domains interacting with d. To that effect, an additional step is introduced; i.e., the source map is first transformed into an intermediate interaction map (MD_{S}) connecting clusters of interaction domains. A correspondence is then built between this intermediary interaction map and the target proteome, and the interactions are inferred along this correspondence. The IDPP method is detailed below and is shown in FIG. 1 along the naive method.

The first step in the Interacting Domain Profile Pair method is to transform the source protein interaction map into an intermediary Domain Cluster Interaction Map. An intermediary domain cluster interaction map MD_{S} is generated from M_{S} using the following procedure.

To construct MD_{S} vertices the clustering of Interacting Domains is first analyzed using I-link clusters (connectivity clusters) and then using S-link clusters (sequence similarity clusters) from the source interaction map. Any other protein information such as a protein expression profile or data from the literature may also be used. The I-link functional linkage is determined by clustering of IDs that interact with the same region of the same partner.

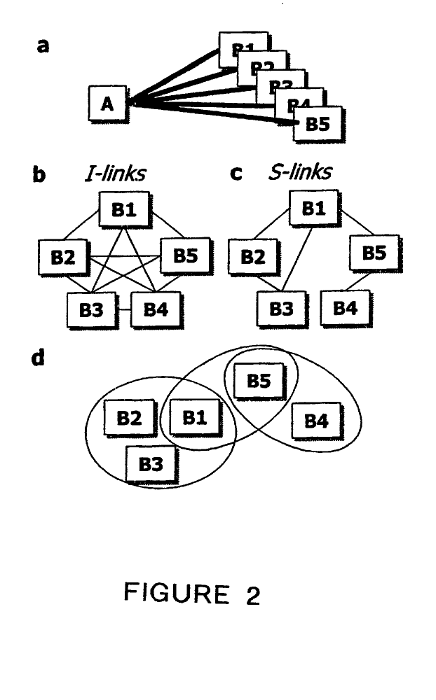

As a first step, domains of different proteins that interact with a common region are clustered. Thus, for each protein x_{S} in P_{S}, the IDs of all proteins interacting with x_{S} are examined. These IDs can be clustered into interacting clusters (IC), where an IC of protein x_{S} is defined as a set of M_{S} IDs interacting with a common region of x_{S}. For a given protein x_{S}, both the number of ICs and the number of IDs within each IC are bounded by the number of proteins interacting with x_{S}. In graph-theoretical terms, an IC can be viewed as a clique (a subgraph where each vertex is linked to all others) where all the IDs are pair-connected by links that means that they interact with the same part of the x protein. Thus, I-links are obtained (See, FIG. 2b).

The clustering of homologous lDs is then undertaken. Within each IC, domains are further regrouped showing high sequence similarity. ID sequences are compared pairwise. Alignments above a chosen threshold; i.e., wherein the Smith-Waterman score (SW) is>60 are considered significant (see Implementation below in the Examples). If domains d₁ and d₂ show a significant similarity, a sequence similarity link (S-link) is generated between d₁ and d₂.

Domains are clustered on the basis of S-links, i.e., a cluster is created for each clique of S-links (FIG. 2 c). This clustering is non-transitive (i.e., a given domain d can be clustered to a cluster C if d shares a significant sequence similarity with all the sequences in C) and non-exclusive (a domain can participate in several clusters). The resulting clusters are in fact cliques both in terms of S-links and of the previously described I-links. These clusters are termed n-SIC (Similarity & Interaction Cliques), where n is the number of IDs in the cluster (1-SIC are degenerated cliques containing a single ID). See, FIG. 2 d wherein a schematic representation of the clustering of ID sequences into n-SIC is shown. The set of vertices of MD_{S} is defined as the set of all n-SICs (n>0).

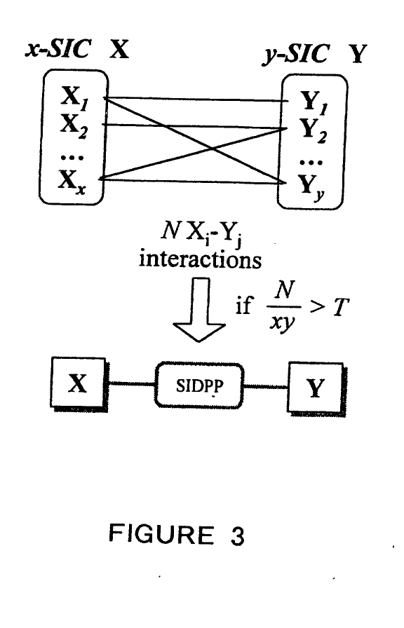

The next step involves the construction of MD_{S} edges that are interactions between domain clusters. Interactions between SICs, called ID Profile Pairs (IDPP), are generated as follows. All possible pairs of SIC are analyzed. A pair of SIC (SIC₁; SIC₂), SIC₁={ID_{1,1}, . . . , ID_{1,n1}} and SIC₂={ID_{2,1}, . . . , ID_{2,n2}}, is said to define a IDPP if the number of (ID_{1,i}, ID_{2,j}) pairs connected in the source interaction map divided by n₁n₂ (the total number of possible ID pairs between SIC₁ and SIC₂) is superior or equal to a threshold T (FIG. 3).

In a perfect world, T would be 100%, meaning that the pair of SIC must be fully inter-connected to create a IDPP. Since the experimentally derived M_{S} is necessarily incomplete with respect to the ideal map of all possible physical interactions in P_{S}, however, pairs with partial but high interconnection should also result in the creation of a IDPP. For example, two 2-SIC inter-connected by “only” 3 interactions (out of 4 possible) should yield a IDPP, since this case appears significant from a biological point of view. The “missing” interaction can most of the time be explained by the non-exhaustiveness of the source map. For example, the threshhold T=75% was chosen as the threshold. However, the threshold can be 50% <T<100%.

Three types of IDPP are distinguished according to the number of elements in each cluster of the pair: ‘1:1’, ‘1:n’, and ‘m:n’, where m and n are strictly greater than 1. To simplify notations, ‘m:n’ IDPP are now referenced as ‘n:n’, even when m is different from n. Intuitively, a IDPP is meant to gather all the evidence from M_{S} about a given domain-domain interaction. IDPPs for which the two SIC are not degenerated (1:n and n:n) can be seen as combining connectivity and sequence similarity information, while degenerated 1:1 IDPP reflect only interactions between single domains.

In the next step a correspondence between MD_{S} and P_{T} is constructed by profile building and searching for similarities between interacting domain profiles in the target proteome P_{T}.

For each n-SIC that contains more than one member (n>1), a profile is built from the multiple alignments of the ID sequences.

If n=2, the alignment is the previously computed pair-wise comparison result,

If n>2, it is recomputed as a multiple alignment. See, for example, Thompson et al, 1994.

Note that by construction, IDs that are members of the same n-SIC share a sequence similarity in a single region. A model profile is then built from the sequence alignment, for example using a Hidden Markov profile or a Gribskov model (Gribskov et al, 1987).

The similarities between domain profiles in target proteome P_{T} are then searched as follows. For each n-SIC, a library containing the target protein sequences is scanned, using as a probe a single ID sequence if n=1, or else an ID profile. Significant hits define homologies between target protein domains and source ID profiles. The correspondence between vertices of MD_{S} and P_{T} is defined by associating to each n-SIC the set of P_{T} protein domains similar to its profile.

The final step involves the inference from MD_{S} to M_{T}; i.e., the prediction of interactions from the IDPP collection. In this step, the property “x interacts with y” is transported along the correspondence; i.e., a property of an object such as a protein is assigned to every object linked to it such as by a significant sequence similarity or an interaction property of a couple A-B of objects such as two proteins or protein domains is assigned to every couple of objects A′-B′ for which A′ is linked to A and B′ is linked to B. This inference step is similar to the one described below in Example 1 for the “naive” method.

The present invention also permits the inference of a Predicted Biological Score or PBS® at the time of inference or deduction of the protein interaction map or PIM®. This PBS® value is indicative of the probability that the inferred protein-protein interaction exists effectively in nature.

The process can also predict multiple interactions of the same protein such as eukaryotic proteins that can modulate and carry many domains involved in many different interactions.

The present invention also concerns the application of motifs of interacting domains. These motifs can be established for all interacting partners and can be multi-organism. One can compare the functional domain motifs to establish the presumed function of the protein or to extrapolate the interactions between other proteins with the same motif. These motifs can be used in the yeast two-hybrid assay to identify new interacting proteins.

The present invention also provides the predicted interaction map, as well as a record of the interaction map that is generated. This record may be in paper, electronic or digital form.

The use of sequence similarity in the present invention on interacting domains rather than full-length proteins reduces the false-positive rate induced by multi-domains proteins. Moreover, the combination of sequence and interaction information allows the identification of ID profiles, “flexible patterns” of sequence correlated to physically interacting structures, that enhance the prediction sensitivity and then reduce the false negative rate. As an additional feature, these profiles also represent new potential binding motifs.

The IDPP method yields several categories of predicted protein-protein interactions, corresponding to different levels of “available evidence” in the reference interaction map. The number of predicted interactions can be modulated according to the biological aim by tuning the stringency parameters of sequence similarity algorithms.

Special emphasis should be placed on the fact that the method relies heavily on the completeness, accuracy and level of detail (definition of protein domains) of the reference data set. Its predictive power should increase as the reference interaction map becomes more complete, as long as special care is taken with quality control of experimental procedures.

Perspectives opened by this work include ab initio prediction of “virtual” protein interaction maps on a variety of organisms related to existing experimental protein interaction maps, combined use of prediction and experimental work to speed-up the construction of new interaction maps, or the identification of new shared interaction domains. Also, as an alternative to additional experimental evidence on a single organism, the biological relevance of using reference protein interaction maps combining interactions from several organisms can be accomplished using the present invention.

The IDPP method not only permits the identification of protein-protein interactions but also defines restricted interacting domains. Such domains can be functionally equivalent to other well-characterized motif based domains such as ATP-binding domains, histidine kinase domains or SH2 domains.

Moreover, the IDPP method can be used to infer a large-scale protein interaction map across organisms, to assess manually the rate of true positives and determine which are the most discriminatory parameters that best predict a biologically meaningful protein-protein interaction. The prediction of protein-protein interaction maps across organisms allows the formulation of new biological hypotheses such as functional assignment and to validate in return some interactions of the original experimental map.

In order to fully illustrate the present invention and advantages thereof, the following specific examples are given, it being understood that the same are intended only as illustrative and in nowise limitative.

### EXAMPLES

Materials

The following materials were used in the examples that follow.

As a source map (M_{S}), the recently published protein interaction map of Helicobacter pylori (Rain, Selig et al. 2001) (http://pim.hybrigenics.com) was used. The map was obtained experimentally by a high-throughput two-hybrid strategy using a random genomic fragment library of the H. pylori strain 26695 (Tomb, White et al. 1997) and specific bioinformatics processes. It is composed of 1524 interactions between 2680 independent interacting domains, called ID. Interacting domain amino acid lengths ranged from 10 to 700 (about 160 on average). Each interaction (that is, a pair of IDs) was scored with a reliability value that allows potential artifacts of the two-hybrid method to be filtered out. Over 1200 interactions yielded a trustable score, thus connecting 46.6% of the 1590 H. pylori putative proteins, representing an average connectivity of 3.36 partners per connected protein (without counting the 62 homodimeric connections).

Escherichia coli K-12 MG1655 was used as a target organism for protein-protein interaction map prediction. Protein sequences and functional categories were downloaded from the E. coli Genome Project homepage (http://www.genetics.wisc.edu, version M52, September, 1997).

Methods (Implementation)

The following implementation methods can be used in the present invention and which were used in the following examples.

The pair-wise ID comparisons (in the IDPP method) were performed with the ssearch33 software application from the FASTA3 program package (Pearson 2000). The different sets of parameters recommended by Pearson were tested, and those with the highest gap opening and extension penalties (respectively −14 and −2) were chosen. The matrix used was BLOSUM50. A significant pair-wise alignment is defined as an alignment with a Smith-Waterman score (SW) greater than a threshold fixed to 60, ensuring enough amino acid similarity on a long-enough region.

Multiple ID alignments (involving more than two sequences) were computed with the CLUSTAL W software application (Thompson, Higgins et al. 1994). Once again, parameters were tuned to minimize the number of gaps (matrix=BLOSUM, gap opening penalty=14, gap extension penalty=2). Hidden Markov model profiles were then built with the hmmbuild software application from the HMMER package (see http://hmmer.wustl.edu/).

The E. coli protein library was screened with ssearch33 in cases where the probe was a single sequence (full-length protein sequences in the naive method, and single ID sequences of 1-SIC in the IDPP method). The hmmsearch software (package HMMER) was used for ID profiles of n-SIC with n>1. Matches below a fixed E-value threshold were considered as significant and defined a homology between the H. pylori probe sequence and the E. coli protein domain sequence. Several E-value thresholds were tested. The results presented here were obtained with a threshold of 1×10⁻⁵, chosen on the basis of examples and in agreement with previous studies (e.g., (Karp, Ouzounis et al. 1996)).

### Example 1

The naive method is a method wherein there is a construction of the correspondence between the protein source P_{S} and the protein target P_{T}. A target interaction map M_{T} is then completed by linking the proteins in P_{T}. The naive method directly screens the target proteome P_{T} with full-length protein sequences of P_{S} and builds a correspondence according to best matches and infers interactions along this correspondence. In the naive method a correspondence between P_{S} and P_{T} is constructed by screening a library of target protein sequences against the full-length sequences of proteins connected in M_{S}. A protein x_{T} of P_{T} is termed homologous to an protein x_{S} of P_{S} if there is a significant similarity between their sequences (See the Implementation section above for details on algorithms and thresholds). The correspondence associates to each protein of P_{S} the set of its homologous proteins in P_{T}.

The target interaction map M_{T} in the naive method is then completed by linking the proteins in P_{T}. An interaction is predicted between two different target proteins x_{T} and y_{T} if there are two different proteins x_{S} and y_{S}, respectively homologous to x_{T} and y_{T}, and interacting in M_{S}. See, FIG. 1. (The Inference Step)

### Example 2

The IDPP method and the naive method were both applied to the inference of an E. coli protein interaction map from the reference H. pylori protein interaction map. The results are summarized in Table 1. Predicted interactions are separated in three main categories according to their origin: IDPP specific (A), predicted both by IDPP and naive methods (B), and specific to the naive method (C). The latter category is further divided into C1 and C2, respectively potential and confirmed false positives of the naive method (see below).

TABLE 1
General features of the predicted protein-protein interaction maps

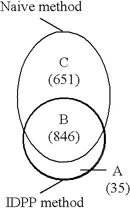

Interaction map

| | | Connected |
|---|---|---|
| | Interactions | proteins |
| Hp (source) | 1524 | 741 |
| Naïve method (total) | 1497 | 543 |
| IDPP method (total) | 881 | 412 |
| A-IDPP-specific | 35 | 40 |
| B-Common IDPP/naive | 846 | 400 |
| C-Naive-specific: | 651 | 310 |
| C1 category | 399 | 160 |
| C2 category | 252 | 150 |

In the first step of the IDPP method, the 1524 interactions connecting 2680 IDs of the original H. pylori interaction map yielded an abstract domain cluster interaction map containing 1568 vertices (n-SICs) (including 214 with n>1), and 1810 IDPPs (edges). Fifty (3%) of these IDPPs are ‘n:n’ pairs, 442 (24%) are ‘1:n’ pairs, and the 1318 (73%) remaining abstract interactions were created from a single pair of IDs from the original interaction map. The correspondence established between this abstract domain interaction map and the E.coli proteome led to 881 interaction predictions, connecting 412 out of the 4290 proteins (9.6%).

Interactions predicted from 1:n and n:n IDPPs are listed in Table 2. These interactions can be seen as “higher-confidence” predictions, as they result from the clustering of information coming from two or more independent interactions of the original map.

TABLE 2
Interactions predicted from 1:n and n:n ID profile pairs

| | Protein | | | |
|---|---|---|---|---|
| Type | interaction | | Category | Comments |
| N:n | GyrA | gyrA | A | gyrA forms an A2B2 complex with |
| | | | | gyrB |
| N:n | Mfd | mfd | A | |
| 1:n | FliS | fliC | B | fliS and fliC are both involved in |
| | | | | flagella |
| 1:n | clpA | relA | B | clpA and clpB are proteases |
| | clpB | spoT | | |
| 1:n | RpoC | relA | B | relA and spoT are involved in the |
| | | spoT | | stringent response. |
| 1:n | UvrA | b0484 | B | |
| | | b3469 | | |
| | | rpoB | | |
| | | rpoC | | |
| 1:n | UvrA | uvrB | B | uvrA, uvrB, and uvrC form a |
| | | | | complex |
| 1:n | UvrB | rpoC | B | |
| 1:n | b0177 | ch60 | B | |
| 1:n | RpsD | fliA | B | fliA, rpsD, rp32, and rpoS are all |
| | | rpoD | | different sigma factors |
| | | rp32 | | |
| | | rpoS | | |
| 1:n | GppA | dcoP | B | |
| 1:n | rep | helD | A | |
| | uvrD | ex5B | | |
| 1:n | UvrD | rep | A | |
| N:n | UvrD | uvrD | B | |
| N:n | Rep | rep | B | |
| 1:n | RplB | dbpA | B | rplB is the ribosomal protein 50S. |
| | | deaD | | dbpA and srmB are involved with |
| | | rhlE | | the 50S ribosomal subunits. |
| | | srmB | | |
| 1:n | top1 | flgB | B | |

As can be seen from the above results, the IDPP method appears to be significantly more stringent than the naive method (651 interactions predicted by the naive method were not confirmed by IDPP), yet yields a number of additional, highly domain-specific, predicted interactions.

The largest interaction category (B) includes the 846 interactions that were predicted by both methods. While IDPP method strongly reinforces the naive prediction in some cases (1:n and n:n pairs), for the majority of these interactions stemming from 1:1 pairs, the IDPP prediction essentially confirms that the correspondence computed with the naive method is compatible with information on the interaction domain, eliminating putative false positives in the process (see category C below).

Category A includes thirty-five interactions that were predicted by the IDPP method but not by the naive method. Among these IDPP-specific predictions, 28 result from the highest selectivity of short ID regions compared to full-length proteins. For instance, HP0422 is a 615 amino acid long protein that shares a similarity with the E. coli lysA; that similarity was not considered significant since the corresponding E-value of 5×10⁻⁵ is greater than the chosen threshold. In contrast, the corresponding HP0422 ID (located between amino acids 141 and 466) shows significant homology to the 108-284 region of lysA (E=5×10⁻⁶).

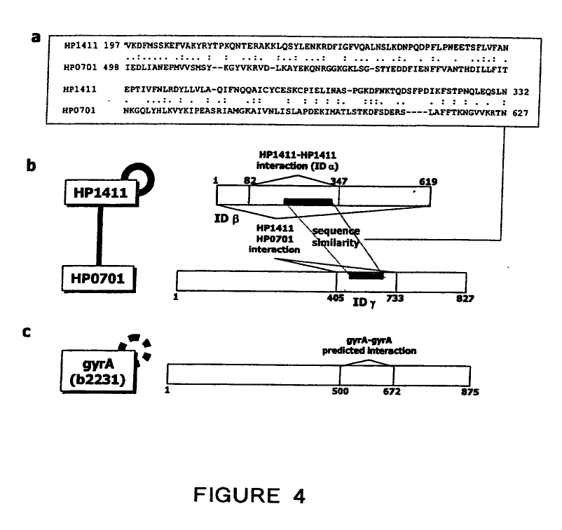

In addition, the use of ID profiles instead of single ID sequences allowed the detection of homologies at lowest levels of sequence similarity. For instance, H. pylori protein HP1411 has no homolog in E. coli, whether one considers its full-length sequence or a ID sub-sequence. Nevertheless, because HP1411 interacts with the gyrA H. pylori protein and also shares a sequence similarity with gyrA, a profile merging gyrA and HP1411 sequences was built and succeeded in selecting the homologous E. coli gyrA protein. A gyrA homodimer was thus predicted in E. coli (FIG. 4). This prediction is confirmed by the SwissProt annotations, according to which gyrA forms an A2-B2 complex with gyrB.

The 651 interactions of category C were predicted by the naive method but not by the IDPP method. The main explanation, confirmed by several manual analyses, reIDes in the difference between global similarity found by using full-length sequences and homology between a ID profile and a protein subsequence.

252 (40%, sub-category C2) of these 651 interactions were predicted through sequence similarity of a region that does not contain the ID and are thus in all likelihood false positives. For example, HP0250 is a 516 amino acid long protein involved in oligopeptide transport. Its N-terminal region (2-243) is very similar to several E. coli other transport ATP binding proteins, including for example artP (E-value=4×10⁻⁷). Since HP0250 interacts with msrA (HP0224) in the H. pylori source map, an interaction between the E. coli msrA protein and artP has been predicted by the naive method. However, the HP0250 ID interacting with msrA is located in the C-terminal region (458-516) and shares no similarity with artP. These are strong indications that HP0250 is a multi-domain protein, and that the predicted msrA-artP is a false-positive interaction of the naive method.

The 399 remaining interactions (sub-category C1) were obtained through sequence similarity that was significant when considering the whole protein but not when considering the shorter included ID region. Clearly, modification of the similarity search algorithms parameters can impact this number by transferring some proteins between this latter category and category B. However, that finer “manual” inspection of the sequence alignment in the vicinity of the ID region and/or additional biological expertise is needed to confirm “false positive” status for these interactions. Pending further validation, they should be considered as putative false positives, or “lower-confidence” predictions.

Three tests against existing functional data were applied to a first “indirect” assessment of the validity of the IDPP approach to interaction prediction.

First, interactions predicted by the IDPP method were analyzed in terms of functional categories for the E. coli K-12 genome (a protein is assigned at most one category in this functional classification), and compared to a theoretical background obtained by random drawing. 505 of the 881 interactions (57%) involved pairs where both proteins had assigned functional categories, which is significantly higher than the 24% background. Among these 505 interactions, 143 (28%) involved proteins assigned to the same functional category: this is also significantly higher than the 8% (p<1^{e}-10) random theoretical background. Interestingly, these 143 proteins were found to be distributed preferentially in seven functional categories: “Transport and binding proteins” (12%), “Translation, post-translational modification” (12%), “Cell processes” (10%), “DNA replication, recombination, modification and repair” (6%), “Transcription, RNA processing and degradation” (6%), “Central intermediary metabolism” (6%), and “Energy metabolism” (6%). One interpretation is that these categories gather functions common to all bacteria.

FIG. 4 illustrates the prediction of gyrA homo-dimerization in E. coli. In the reference protein interaction map, ID β of HP1411 interacts with ID γ of HP0701 and HP1411 interacts with itself through ID α (b). When the IDPP method is applied:

ID α and ID γ are clustered in the same IC since they both interact with the same region of HP1411 (b).

ID α and ID γ are then clustered in the same 2-SIC, since the 197-332 region of HP1411 and the 498-627 region of HP0701 are similar (103 amino acid overlap, 32% of identity, (a)).

This leads to the creation of a ‘homodimer’ 2:2 ID profile pair connecting the 2-SIC with itself. When used as a probe to screen a E. coli protein sequence library, the 2-SIC profile selected a 172 amino acid long domain on the gyrA protein, and gyrA was predicted to interact with itself through this domain (c).

In a second validation test, for each interaction predicted by the IDPP method, SwissProt annotation keywords of both partner proteins were retrieved, and common keywords were counted, after discarding irrelevant keywords such as “hypothetical protein,” “3D-structure,” or “transposable element.” Among the 351 interactions for which both proteins were annotated, the average number of common keywords was estimated to 0.4. To obtain a rough estimate of the background noise, the same keyword retrieval procedure was performed for a set of random pairs of annotated E. coli proteins and resulted in an average of 0.2 shared keywords per pair (p<1×10-5).

Finally, the predicted interactions against physical location of genes in the genome were assessed. The organization of the E. coli genome into operons suggested a functional link between corresponding gene products. Table 3 lists the interactions predicted by the IDPP method between two proteins encoded by genes in the same genomic region.

TABLE 3
Predicted interactions between products of “neighbor” genes

| | |
|---|---|
| Protein 1 | Protein 2 |

| | | | |
|---|---|---|---|
| b0094 | ftsA | b0095 | ftsZ |
| b1079 | flgH | b1080 | flgl |
| b1885 | tap | b1887 | cheW |
| b1886 | tar | b1887 | cheW |
| b1887 | cheW | b1888 | cheA |
| b1923 | fliC | b1925 | fliS |
| b2221 | atoD | b2222 | atoA |
| b3313 | rplP | b3317 | rplB |
| b3985 | rplJ | b3986 | rplL |
| b4200 | rpsF | b4202 | rpsR |

### Example 5

According to this example, the IDPP method was applied to infer a large-scale protein interaction map across organisms and to assess manually the rate of true positives of such method, as well as determining which are the most discriminatory parameters that best predict a biologically meaningful protein-protein interaction.

Assessment of Prediction Validity

Each predicted interaction was manually evaluated by comparing the annotations in public databases [SWISS-PROT (Bairoch and Apweiler, 2000) and Colibri (Medigue et al. 1993) databases], checking reference literature (Neidhardt, 1996), and original literature of each protein partner. The “++” category represented interactions identified in the literature and documented in databases. They could be considered as true-positives of the prediction method. Interactions classified in the “+” category were not strictly described but made biological sense. Interactions in the “−” and “− −” categories were judged to make improbable and very improbable biological sense, respectively (for instance the two protein partners were differentially located inside the cell or belonged to completely unrelated pathways). The last category (‘?’) regrouped interactions for which at least one of the partners has an unknown function and no similarity with other characterized protein domain.

As set forth in Example 2, the protein-protein interaction map of Escherichia coli was predicted from the interaction map of the human gastric pathogen Helicobacter pylori (Rain et al., 2001) by using the IDPP method and a simplified “naïve” method (Wojcik and Schächter, 2001). E. coli was an ideal target organism to assess the IDPP method due to our extensive current knowledge compared to other organisms. As illustrated in Table 1, predictions from both methods were pooled and separated in three main categories according to their origin: IDPP specific (35 interactions), predicted both by IDPP and “naïve” methods (846 interactions), and specific to the “naïve” method (651 interactions). Predictions specific of the ‘naive’ method for which the interacting domains were not contained in the homologous sequence regions that permitted to transfer the interaction were confirmed to be false positives (Wojcik and Schächter, 2001) and were removed from the analysis (252 interactions), thus leading to a set of 1280 inferred interactions, already suggesting a higher selectivity of the IDPP method. The resulting protein interaction can be visualized through the PIM Rider® software platform at http://pim.hybrigenics.com.

The predicted interactions were manually assessed one by one in order to assign a category to each interaction (Table 4).

B. Predictive Discriminant Analysis

Several variables of the IDPP prediction method were binary-coded by assigning the 1 value to ranges of values that a priori favored true-positive predictions (Table 4). There were two types of variables: those that were properties of the interaction itself (marked with a # in the Table 4), and those that were properties of the interacting protein domains. In this latter case, there were always two values per variable (one for each partner). Because the aim of the discriminant analysis was to eliminate false-positive predictions, only the a priori most unfavorable value was used. Discriminant analysis was run using the XIStat software package (http://www.xIstat.com), using the hypothesis of equality between variance and co-variance matrices.

TABLE 4
Discriminant variables

| | Binary-encoding | | |
|---|---|---|---|
| Discriminant variable | 0 | 1 | Correlation |
| PBS value of source interaction | B, C, or D | A | 0.78 |
| (a)(#) | | | |
| First rank in the homology score | no | yes | 0.49 |
| Single homologous domains | no | yes | 0.34 |
| identified | | | |
| Naive method-specific prediction | yes | no | 0.24 |
| (b)(#) | | | |
| Size of the largest interacting | >1000 bp | <1000 bp | 0.19 |
| domain | | | |
| High connectivity in the source | E PBS | not E PBS | −0.17 |
| network (a)(#) | | | |
| Number of source interactions | 1 | >1 | 0.13 |
| (c)(#) | | | |

In Table 4, the annotations (a), (b), (c) and # mean the following:

(a) the PBS score was split into two different variables: one representing the local reliability of the interaction, and the other taking into account the effect of highly-connected prey in the two-hybrid interaction map (Rain et al. 2001);

(b) see (Wojcik and Schächter, 2001) for details;

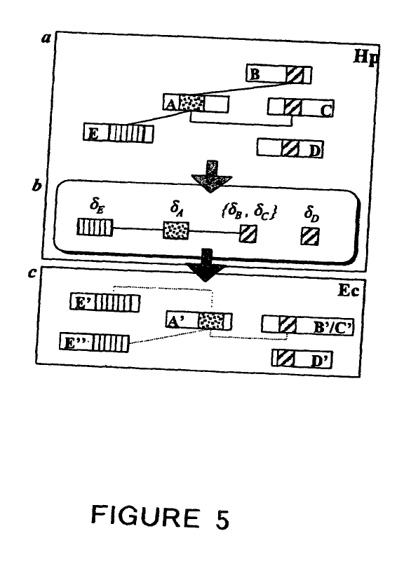

(c) in case of inference through domain profiles (FIG. 5), one prediction could result from several source interactions; and

(#) these parameters are variables of the interactions rather than individual protein partners

TABLE 5
Classification of predicted interactions according to
estimated biological sense

| | Predicted | Predicted | |
|---|---|---|---|
| Category (a) | Homodimers | heterodimers | Total |
| ++ | 54 | 40 | 94 |
| + | 10 | 48 | 58 |
| − | 9 | 44 | 53 |
| −− | 11 | 562 | 573 |
| ? | 47 | 455 | 502 |
| Total | 131 | 1149 | 1280 |

In Table 5, the “++” category gathers literature confirmed interactions; the “+” category means interactions that make biological sense; the “−” and “−−” interactions (between known proteins) means that they are biologically meaningless; the questionable “?” category means interactions between non-annotated proteins.

For well-characterized protein partners, interactions were clustered into four categories, ranging from validated interactions (“++”) to the category grouping interactions that make very improbable biological sense (“−−”). An additional questionable category (“?”) grouped interactions for which it was unable to assess the biological validity from literature. The “+” and “++” categories corresponded to 152 predictions, 12% of the total number of predictions. The “−” and “− −” categories gathered 606 heterodimeric predictions that were estimated to be biologically meaningless based on the current available literature on E. coli. It appeared to represent 53% of false-positives of the prediction method. However, these categories were probably over-estimated since it was difficult to state definitely that an interaction did not occur. For instance, some gene products appeared as completely unrelated but interacted together upon selective pressure (Wagner, 2000), and the existence of alternative metabolic routes has been evidenced (Edwards and Palsson, 1999). Thus, the 12% prediction rate should be taken as the very minimum true-positive frequency as assessed given the current biological knowledge. This compared to estimations made on experimental data on the yeast proteome (Uetz, et al. 2000). Additionally, one should note that these results were independent from and did not take into account the reliability of the yeast two-hybrid interactions of the source map (H. pyloi map).

The 1280 interactions in E. coli were predicted from 367 different “source” interactions (45 homodimers and 322 heterodimers) out of 1524 in the original H. pylori interaction map (24%). Homodimers were kept away of further study because they represented particular cases both from technical and biological points of view. An intra-molecular interaction could indeed be transferred into inter-molecular interactions. For instance, the intra-molecular interaction between regions 1 and 4 of the sigma factor RpoD in H. pylori was inferred into inter-molecular interactions between the E. coli sigma factors (RpoD, RpoN, RpoH, and FIiA). Homodimeric predictions required thus additional manual analyses.

TABLE 6
Correspondence between source and inferred interactions

| | | | Category | | |
|---|---|---|---|---|---|
| | Source | Inferred | propensities (a) | | |
| PBS | interactions | interactions | ++/+ | ? | −/−− |
| A | 71 | 246 | 2.3 | 1.1 | 0.5 |
| B | 49 | 203 | 0.3 | 0.9 | 1.3 |
| C | 21 | 79 | 0.4 | 0.9 | 1.2 |
| D | 140 | 537 | 0.6 | 1.0 | 1.1 |
| E | 41 | 84 | 1.3 | 0.9 | 1.0 |
| Total | 322 | 1149 | | | |

In Table 6, the propensity of a prediction with a given category (e.g., “++”) to come from a source interaction yielding a given PBS (e.g., “A”) is the ratio between the frequency of the category in the PBS class and its frequency in the whole data set. For instance:

P(“++”,A)=n(A∩“++”)/n(A)n(”++”)/n_{TOT}

The categories “++” and “+” on one side, and “− −” and “−” on the other side, are merged.

Table 6 illustrates the correspondences between source and inferred heterodimeric interactions depending on the reliability value assigned to each source interaction, the PBS® (Rain et al. 2001). The PBS values were clustered into four categories, from A (very reliable) to D (probably artifact). A fifth category, E, gathered interactions with highly connected proteins for which it was a priori impossible to distinguish between two-hybrid artifacts (prey proteins that were non-specifically selected in numbers of independent screens) and interactions with biologically highly connected proteins (e.g., chaperones). On average 3.6 interactions were predicted in E. coli per source interaction in H. pylori, irrespective to the PBS value. This number was compared to the 2.7 fold ratio between E. coli and H. pylori proteome sizes (4290 and 1590 coding sequences, respectively). However this average ratio hid dissimilarities: 133 source interactions (41%) produced 133 predictions (11%) while twelve source interactions (4%) induced 395 predictions (34%). This asymmetry appeared to be due to certain well-defined interacting domains that could be considered as true functional domains (see the interactions MCPs-CheW and CheA-CheY family of response regulators illustrated in FIG. 5). The literature-confirmed predictions (“++” and “+”) were favored in the PBS A class (that is, being inferred from a source interaction that was biologically highly probable) as opposed to the “−” and “− −” categories. The questionable category predictions were equally distributed over PBS classes.

C. Discriminant Prediction Parameters

Therefore, several parameters of the IDPP method prediction were binary-encoded and assessed by a predictive discriminant analysis using the 602 heterodimeric interactions of the “++” and “− −” categories as a reference set. Table 4 lists the chosen parameters and the correlation between the discriminant variables and the initial variables, indicating the weight of each parameter (ranging from −1 to +1; a positive correlation indicates a variable favorable to true-positive predictions with respect to the chosen encoding). The PBS value was the main discriminant variable to separate true-positives from false-positives (correlation factor c=0.78), thus confirming the pertinence of scoring the source interaction reliability in order to infer true-positives. The second most discriminant variables were the rank of homology (c=0.49) and the number of homologous proteins (c=0.34) for each interacting partners. It confirmed that i) the first ranked homologous protein appeared to be an orthologue in terms of binding ability, and ii) if there was only one homologue below the fixed homology score threshold, it was probably an orthologue. On the other hand, an interaction not specifically predicted by the ‘naive’ method had a low correlation factor (c=0.23) contrary to expectations. The relative small advantage of an IDPP predicted interaction did not reflect entirely the reality and was biased due to the automatic exclusion of 252 predicted interactions specific of the ‘naive’ method confirmed previously to be false-positives.

D. Assignment of an Inference Score

The whole set of predicted heterodimers was then classified according to the optimized discriminant factors (Table 7).

TABLE 7
Prediction of true-positives by discriminant analysis

| Category | Total | Predicted | True-positives (d) |
|---|---|---|---|
| ++ (e) | 40 | 36 | 90% |
| + | 48 | 14 | 29% |
| − | 44 | 22 | 50% |
| −− (e) | 562 | 93 | 16% |
| ? | 346 | 100 | 29% |
| Total | 1149 | 266 | 23% |

In Table 7, the annotations (d) and (e) have the following meaning:

(d) Predictions yielding an inference score greater than 0.5; and

(e) These two categories were used as the reference set of the discriminant analysis.

An inference score, ranging from 0 to 1, was assigned to each prediction: it represented the probability to be a true-positive prediction. The error rate of reclassification of “++” and “−−” category prediction into predicted true-positives and false-positives was 16%: ninety three “−−” predictions out of 562 were predicted to be true-positives and 4 “++” predictions out of 40 to be false-positives. Among the 100 questionable predictions classified in the true-positive category (Table 7), eighty four involved a characterized gene product with a protein of unknown function, thus straightforwardly proposing a functional assignment for the latter. For instance the gene product YhbC (b3170) was predicted to interact (inference score>0.99) with the ribosomal sub-unit RpIP (b3313). This interaction was inferred from a very reliable source interaction (PBS A, found in two reciprocal two-hybrid screens) between the RplP-homologous ribosomal protein RpI16 (HP1312) and the conserved hypothetical HP1046 protein. Furthermore, both genes coding YhbC in E. coli and HP1046 in H. pylori are organized in operons with other genes coding for proteins involved in ribosome assembly and translation. HP1046 has two upstream genes rbfA and infB coding for ribosomal-binding factor A (HP1047) and the translation initiation factor IF-2 (HP1048), respectively. In E. coli, downstream of yhbC, nusA, inIB, rbfa and rpsO are found, all genes also coding for proteins involved either in ribosome assembly or translation. Taken all together, YhbC and HP1046 appeared to be involved in ribosome assembly. There were 8 interactions predicted between proteins for which the genes belonged to the same operon, as defined in the RegulonDB database (Salgado et al. 2001). Seven of them were from a PBS A source interaction, were classified into the “++” category, and yielded an inference score greater than 0.94. The eighth prediction came from a B PBS interaction, was in the “+” category, and yielded an inference score of 0.76.

E. Application to a Biological Pathway

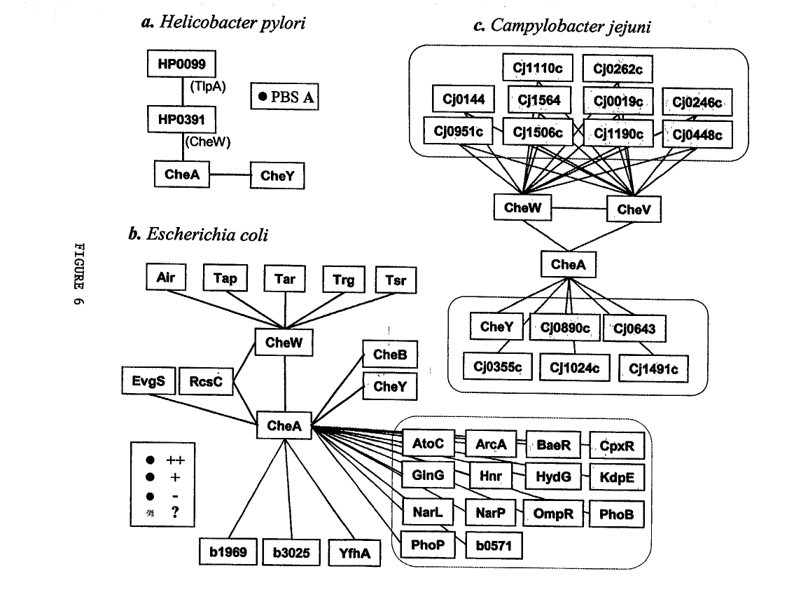

This analysis was taken a step further by inferring the protein-protein interaction map of Campylobacter jejuni from the source interaction map of H. pylori. The FIG. 6 illustrates a typical example where we compare some source interactions with the corresponding predicted interactions in E. coli and C. jejuni. We chose a source interaction network that included only highly reliable yeast two-hybrid interactions (PBS A) to maximize the predictive nature of the inferred interactions. In H. pylori, a network was identified between the methyl-accepting chemotaxis (MCP) transmembrane sensory protein TIpA (HP0099), CheW (HP0391), CheA (HP0392) and CheY (HP1067). The inferred interaction network in E. coli corresponds to already well-characterized interactions of the chemotaxis regulatory network (Neidhardt, 1996; Falke et al. 1997). Remarkably, the method predicted a CheA-CheB interaction, absent in H. pylori, reinforcing the improved predictive accuracy of the IDPP method. The C. jejuni inferred network was similar to the E. coli network, although there were some notable differences. Although C. jejuni has a CheB homologue named CheB′ (Cj0924c) the method did not predict an interaction with CheA in C. jejuni. This result was particularly reassuring since Cj. CheB′ appeared to be a truncated version of CheB proteins lacking the CheA-interacting domain, raising the question of its biological role in C. jejune. A ‘naïve’ method would have still predict an interaction based on sequence similarity of the full-length Cj. CheB′ protein. Another major difference concerns the complex network of CheV with all the MCP sensory homologues of C. jejuni, Cj. CheW and Cj. CheA. CheV proteins were first characterized in Bacillus subtilis and were predicted to modulate the chemotaxis response (Rosario et al. 1994), probably by interacting with the same partners as it was found in the inferred interacting network of C. jejuni. Finally, both inferred networks predicted interactions between CheA and a variety of response regulators from the CheY family. Interestingly, crosstalk between CheA and GInG has been described (Ninfa et al. 1988), raising the intriguing possibility that extensive crosstalk phenomena occur in vivo to modulate and fine-tune an organism's response to environmental stimuli and contribute to the robustness of bacterial chemotaxis (Alon et al. 1999).

A direct consequence of defining interacting domains concerns the extent to which two homologous proteins from different organisms can be annotated as orthologues. Although orthology is mainly based on phylogeny it also implies conservation of function. A given protein function results from a combination of several biochemical and structural properties. Given our definition of protein interactions based on domains, these functional domains contribute to the protein function. Therefore, loss or gain of an interacting domain between homologous proteins will inevitably alter part of their function. Cj. CheA and Ec. CheA are classically considered to be orthologues. Nevertheless, only Cj. CheA was predicted to form a dimer or oligomer (see FIG. 6). Analysis of the interacting domains that predict such dimer revealed that Cj. CheA and Ec. CheA have different N-terminal regions. Indeed, Cj. CheA has a CheY homologous N-terminus that was predicted to interact with Cj. CheA histidine kinase domain through either an intra- or an inter-molecular interaction. This is reminiscent of Rhizobium meliloti CheA, indicating that the N-terminal CheY region of Cj. CheA function as a phosphate ‘sink’ (Neidhardt, 1996; Falke et al. 1997) to modulate the state of phosphorylation of Cj. CheY and therefore the chemotaxis response. In E. coli, the degree of phosphorylation of CheY is regulated by a different mechanism involving the phosphatase CheZ (Neidhardt, 1996; Falke et al. 1997). Cj. CheA and Ec. CheA were not entirely functionally equivalent and therefore could neither be considered as true orthologues nor as paralogues. The boundaries allowing the definition of orthologues and paralogues became rather gradual, introducing the notion of partial orthologues. Cj. CheA was considered as a partial orthologue due to a gain of function while the Cj. CheB′ example is due to a loss of function (see above). This notion has direct consequences for functional annotations of genomes. Functional assignments based solely on phylogeny will induce a high rate of false functional predictions. Combining the IDPP method in such predictive algorithms using experimentally defined interacting domain profile pairs will increase the accuracy of such functional assignments.

F. Retro-Validation of Experimental Maps

Finally, the inference of protein-protein interaction maps can be used to add value to the experimental source interaction map in return, both by rescuing false-negatives and filtering out false-positives, especially when the source organism is poorly annotated and the target organism is a well-studied organism. The PBS E category of the original interaction map represents interactions automatically filtered out because one of the domain partners was selected as a prey in a number of independent two-hybrid screens above a fixed threshold and was then considered as probable two-hybrid false-positives (Rain et al. 2001). Some of these interactions with highly connected proteins may however be biologically meaningful. In the present study, interactions of the E category in H. pylori that were inferred into biologically meaningful interactions in E. coli were found (data not shown). Among the 244 interactions yielding a PBS E value in the H. pylori map (12), seven could be “rescued” by an automated inference followed by manual validation. The same reasoning applies to poorly reliable source interactions (PBS D). Moreover source interactions inferred only to biologically meaningless interactions may correspond to undetected experimental false-positives. For example,13 of the source interactions yielding a PBS A value in the source interaction map indicating only ‘−−’ predictions. Three explanations are possible: i) one of the protein function in the source interaction was completely lost during evolution (this protein has only partial orthologues or paralogues in E. coli, as the CheB′ example in C. jejuni, see FIG. 6). Gene fusion events or duplication could similarly interfere in the inference process; ii) the source interaction was a false positive of the two-hybrid system; iii) the predictions were indeed real true-positives but were not yet referenced in the literature. Whenever it was possible to discriminate between these three hypotheses (involving independent validation methods), the inference process could be used to “retro-filter” false-positives out of an experimental protein interaction map.

As set forth in the above examples, the first protein-protein interaction map inference process was assessed by an independent and exhaustive analysis of the literature. The prediction rate was evaluated at 12% at least. The lack of reference data, even on a model organism like E. coli, forbade to better assess the true-positive rate and to evaluate the false-positive proportion. It was however evidenced that the quality of the source interaction map, both in terms of interaction reliability scoring and interacting domain definition, increased the likelihood of true-positive predictions. Nevertheless, the IDPP method showed an improved means to assign new biological functions that will be increasingly useful with the availability of other genome wide interaction maps. This work relied on the underlying notion of homology restricted to a domain of a protein introducing the concept of partial orthologues and viewing the binding ability as a particular function of such domains. This permitted to avoid false-positive predictions due to global homology considerations on multi-functional proteins, which is a critical point when the method is applied to prediction of interaction maps of eukaryotes.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

### References

1. Alon, U., Surette, M. G., Barkai, N., and Leibler, S. (1999) Nature 397(6715):168-71.

2. Altschul, S. F., Gish, W., Miller, W., Myers, E. W. & Lipman, D. J. (1990) “Basic local alignment search tool”, J. Mol. Biol. 215:403-410.

3. Apweiler, R., Attwood T K, Bairoch, A. Bateman, A. Birenery, E., Biswas, M., Bucher, P., Cerutti, L., Corpet, F., Croning, M D., Durbin, R., Falquet, L., Fleischmann, W., Gouzy, J., Hermjakob, H., Hulo, N., Jonassen, I., Kahn, D., Kanapin, A., Karavidopoulo, Y., Lopez, R., Marx, B., Mulder, N J., Oinn, T M., Pagni, M., Servant, F., Sigrist, C J., Zdobnov, E M., “InterPro-an integrated documantation resource for protein families, domains and functional sites” Bioinformatics (2000) Dec. 16 (12): 1145-1150.

4. Bansal, A. K. (1999). “An automated comparative analysis of 17 complete microbial genomes.” Bioinformatics 15(11): 900-8.

5. Bairoch, A., and Apweiler, R. (2000) Nucleic Acid Res. 28(1): 45-8.

6. Bork, P., T. Dandekar, et al. (1998). “Predicting function: from genes to genomes and back.” J Mol Biol 283(4): 707-25.

7. Dandekar, T., B. Snel, et al. (1998). “Conservation of gene order: a fingerprint of proteins that physically interact.” Trends in Biochemical Sciences 23(9): 324-8.

8. Edwards, J. S., and Palsson, B. O. (1999) J. Biol. Chem. 274(18):17410-16.

9. Eisen, M. B., P. T. Spellman, et al. (1998). “Cluster analysis and display of genome-wide expression patterns.” Proc NatI Acad Sci U S A 95(25): 14863-8.

10.Eisenberg, D., E. M. Marcotte, et al. (2000). “Protein function in the post-genomic era.” Nature 405(6788): 823-6.

11. Enright, A. J., I. iliopoulos, et al. (1999). “Protein interaction maps for complete genomes based on gene fusion events.” Nature 402(6757): 86-90.

12. Falke, J. J., Bass, R. B., Butler, S. L., Chervitz, S. A., and Danielson, M. A. (1997) Annu. Rev. Cell Dev. Biol. 13:457-512.

13. Fellenberg, M., K. Albermann, et al. (2000). “Integrative analysis of protein interaction data [In Process Citation].” Ismb 8:152-61.

14. Flajolet, M., G. Rotondo, et al. (2000). “A genomic approach of the hepatitis C virus generates a protein interaction map.” Gene 242(1-2): 369-379.

15. Fromont-Racine, M., A. E. Mayes, et al. (2000). “Genome-wide protein interaction screens reveal functional networks involving Sm-like proteins.” Yeast 17(2): 95-110.

16. Fromont-Racine, M., J. C. Rain, et al. (1997). “Toward a functional analysis of the yeast genome through exhaustive two-hybrid screens [see comments].” Nature Genetics 16(3): 277-82.

17. Gribskov, M., McLachlan, A. & Gisenberg, D. (1987) “Profile analysis: detection of distantly related proteins” Proc. Natl. Acad. Sci. USA. 84:4355-4358.

18. Ito, T., K. Tashiro, et al. (2000). “Toward a protein-protein interaction map of the budding yeast: A comprehensive system to examine two-hybrid interactions in all possible combinations between the yeast proteins [In Process Citation].” Proc Natl Acad Sci U S A 97(3): 1143-7.

19.Karp, P. D., C. Ouzounis, et al. (1996). “HinCyc: a knowledge base of the complete genome and metabolic pathways of H. influenzae.” Ismb 4:116-24.

20.Marcotte, E. M., M. Pellegrini, et al. (1999). “Detecting protein function and protein-protein interactions from genome sequences.” Science 285(5428): 751-3.

21.Marcotte, E. M., M. Pellegrini, et al. (1999). “A combined algorithm for genome-wide prediction of protein function [see comments].” Nature 402(6757): 83-6.

22. McCraith, S., T. Holtzman, et al. (2000). “Genome-wide analysis of vaccinia virus protein-protein interactions.” Proc Natl Acad Sci U S A 97(9): 4879-84.

23.Medigue, C., Viari, A., Henaut, A., and Danchin, A. (1993) Microbiol. Rev. 57(3):623-54.

24. Neidhardt, F. C. (1996) Escherichia coli and Salmonella—Cellular and Molecular Biology, 2nd Ed., ASM Press, Washington, D.C.

25.Ninfa, A. J., Ninfa, E. G., Lupas, A. N., Stock, A., Magasanik, B., and Stock, J. (1988) Proc. Natl. Acad. Sci. USA 85(15):5492-96.

26.Overbeek, R., M. Fonstein, et al. (1999). “The use of gene clusters to infer functional coupling.” Proceedings of the National Academy of Sciences of the United States of America 96(6): 2896-901.

27. Pearson, W. R. (2000). “Flexible sequence similarity searching with the FASTA3 program package.” Methods Mol Biol 132:185-219.

28.Pellegrini, M., E. M. Marcotte, et al. (1999). “Assigning protein functions by comparative genome analysis: protein phylogenetic profiles.” Proceedings of the National Academy of Sciences of the United States of America 96(8): 4285-8.

29. Rain, J. C., L. Selig, et al. (2001). “The protein-protein interaction map of Helicobacter pylori.” Nature 409:211-216.

30.Rosario, M. M., Fredrick, K. L., Ordal, G. W., and Helmann, J. D. (1994) J. Bacteriol. 176(9):2736-39.

31. Salgado, H., Santos-Zavaleta, A., et al. (2001) Nucleic Acids Res. 29(1):72-4.

32. Schwikowski, B., P. Uetz, et al. (2000). “A network of protein-protein interactions in yeast [In Process Citation].” Nat Biotechnol 18(12): 1257-61.

33. Szabo et al., (1995), Curr Opin Struct Biol 5:699-705.

34.Thompson, J. D., D. G. Higgins, et al. (1994). “CLUSTAL W: improving the sensitivity of progressive multiple sequence alignment through sequence weighting, position-specific gap penalties and weight matrix choice.” Nucleic Acids Res 22(22): 4673-80.

35.Tomb, J. F., O. White, et al. (1997). “The complete genome sequence of the gastric pathogen Helicobacter pylori [see comments] [published erratum appears in Nature 1997 Sep 25;389(6649):412].” Nature 388(6642): 539-47.

36.Uetz, P., L. Giot, et al. (2000). “A comprehensive analysis of protein-protein interactions in Saccharomyces cerevisiae.” Nature 403: 623-27.

37. Wagner, A. (2000) Nature Genetics 24:355-61.

38.Walhout, A. J., R. Sordella, et al. (2000). “Protein interaction mapping in C. elegans using proteins involved in vulval development [see comments].” Science 287(5450): 116-22.

39.Walhout, A. J. M. and M. Vidal (2001). “Protein interaction maps for model organisms.” Nature Reviews, Molecular Cell Biology 2: 55-62.

40.Wojcik, J., and Schächter, V. (2001) Bioinformatics 17(Suppl. 1):S296-S305.

41.Xu et al (1996) “The Bioluminescence Resonance Energy Transfet (BRET) system application to interacting circadian clock proteins” PNAS 1:151. All non-patented websites cited in the present specification are incorporated herein by reference.

What is claimed is:

1. A method for obtaining a predicted protein-protein interaction map across organisms, comprising:
(a) creating an intermediary domain cluster interaction map;
(b) searching for similarities between the cluster for each selected interacting domain cluster and in a target organism;
(c) creating a correspondence between the intermediary domain cluster interaction map and the target organism from the similarities; and
(d) predicting a target protein-protein interaction map along the correspondence.

2. The method of claim 1 further comprising (e) building a profile for each selected interacting domain cluster from the intermediary domain cluster interaction map.

3. The method of claim I wherein the clustering is non-transitive and non-exclusive.

4. The method of claim I wherein the intermediary domain cluster interaction map is generated from at least one of a connectivity link (I-link) and a sequence similarity link (S-link) taken from at least one of a source organism map, a protein expression profile and an art annotation.

5. The method of claim 4 wherein said S-link clusters and I-link clusters resulting from step (a) are similarity and interaction cliques, respectively.

6. The method of claim 5 further comprising (f) further analyzing the clusters of similarity and interaction cliques to find interacting domain profile pairs (IDPP).

7. The method of claim 6 wherein said pairs of similarity and interaction cliques (n-SIC) are defined as (SIC₁; SIC₂), SIC₁={ID_{1,1}, . . . , ID_{1,n1}} and SIC₂={ID_{2.1}, . . . , ID_{2,n2}}, and defines an IDPP if the number of (ID_{1,i}, ID_{2,j}) pairs connected in the source interaction map divided by n₁n₂ (the total number of possible ID pairs between SIC₁ and SIC₂) is superior or equal to a threshold T of between about 50% and 100%.

8. The method of claim 2 wherein the profile is built when each sequence and interaction clique contains more than one member from a multiple sequence alignment of interacting domain sequences.

9. The method of claim 8 wherein the sequence alignment is a previously computed pairwise comparison if n=2 or if n>2 said sequence alignment is computed as a multiple sequence alignment.

10. The method of claim 8 wherein a Hidden Markov profile is built from said sequence alignment.

11. The method of claim 9 wherein a Hidden Markov profile is built from said sequence alignment.

12. The method of claim 1 wherein said searching of (b) is performed by using a single interacting domain sequence if n=1, or by using an interacting domain profile if n>1.

13. The method of claim 1 wherein the correspondence in (c) is performed by associating to each n-similarity and interacting cliques (n-SIC) a set of target protein domains similar to said n-SIC profile.

14. The method of claim 1 wherein a predicted biological score (PBS®) is provided with the predicted target protein-protein interaction map.

15. The method of claim 2 wherein the profile of interacting domains is a flexible sequence pattern correlated to physically interacting structures.

16. The method of claim 15 wherein the flexible sequence pattern represents new binding motifs.

17. A protein-protein interaction map obtained by the method of claim 1.

18. A record of the protein-protein interaction map of claim 17 in electronic, paper or digital form.

19. A method of predicting a target organism protein interaction map from a source organism protein interaction map, comprising:
(i) comparing each target organism protein sequence with each source organism protein; and
(ii) transporting an interacting property of two source organism proteins along two target organism proteins showing significant similarities with said two interacting source proteins.

20. A method for predicting a target organism protein interaction map from a source organism protein interaction map, comprising comparing each target organism protein sequence with each interacting domain of a source organism protein specifically involved in an interaction.
